(19) United States
(12) Patent Application Publication
(10) Pub. No.: US 2010/0010339 A1
(43) Pub. Date: Jan. 14, 2010

(54) METHOD AND DEVICE FOR EASY ACCESS TO SUBINTIMALLY IMPLANTED VASCULAR ACCESS PORTS

(76) Inventors: Christopher K. Smith, Columbia, SC (US); John R. Ross, Bamberg, SC (US)

Correspondence Address: PASSE' INTELLECTUAL PROPERTY, LLC, 1717 BRASSFIELD RD., RALEIGH, NC, 27614, US

(21) Appl. No.: 12/561,006

(22) Filed: Sep. 16, 2009

Related U.S. Application Data
(63) Continuation-in-part of application No. 12/047,445, filed on Mar. 13, 2008.

(51) Int. Cl. A61B 6/12; A61M 31/00

(52) U.S. Cl. 600/424; 604/288.01

(57) ABSTRACT

The present invention relates to a novel vascular access port for administering chemotherapeutics and the like that are subintimally implanted and attached to the fascia. By inclusion of one or more UV fluorescing polymers in the port a healthcare worker can shine a UV light in the area of the port and use the fluorescing polymer to find the injection site.

This application is a continuation-in-part of U.S. patent application Ser. No. 12/047,445 field on Mar. 13, 2008 and is included in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to improved vascular access ports. In particular the present invention relates to a vascular access port designed for subintimal implantation wherein at least a portion of the port is made from a UV fluorescing polymer which allows the user to see the port during access use.

2. Description of Related Art

A vascular access port is a vascular implantable port that is designed for subintimally implantation. It is designed for repeated access to the vasculature, for example, for administration of a desired product by injection. Typically, a vascular access port is fixed in position by suturing to underlying fascia in the desired location. Both single and dual access ports (or multiple devices) are frequently utilized on a patient. These devices are made of polymers or metals such as a polysulfone, an acetal plastic or titanium.

Typically they are used for a patient requiring repeated access of the vascular system for delivery of medications, nutritional supplementation, fluids, blood, blood products, sampling of blood and the like. Where dual access ports are used, they are for combination therapy, simultaneous infusions, withdrawal of body fluids and bolus delivery during continuous infusion. A number of commercially available versions are currently sold, for example, the “Vortex” sold by Angiodynamics Inc.

These types of ports typically have a number of problems in their use including problems associated with finding the device. Typically, rubber or other material type beads are placed on the outside a top of the port to aid a technician in finding the port under the skin.

While the use of these ports substantially improves the repeated access problems to the vasculature, it is clear that ports have problems associated with their use. Injection into the port is typically accomplished by the nursing staff or worse lower level medical technicians without the ability to read ultra sound or other techniques for finding the port to access with a needle. Accordingly, it is typical that these technicians and staff thus use either touch or a previously done “diagram” to place the needle. Because of the location and the like of the port these personnel are almost attempting to access the port blindly. Stick site errors result in the patient presenting complications such as pseudoaneursms, aneurysms, thrombus, clots and blockage with the possibility of total occlusion of the graft needing replacement. This is not to mention the potential pain and discomfort the patient experiences.

The resulting complications cost tens of millions of dollars in invasive treatments to cure these problems. That doesn't include lost work time and the problems associated with further surgical intervention. The port recipients who are on public medical assistance and have these ports and end up with these problems not only a have a huge financial burden but also can cost the US taxpayer.

Accordingly, it would be useful if there were additional methods, ports or the like that would aid the healthcare worker in accessing subintimal vascular access ports.

### BRIEF SUMMARY OF THE INVENTION

In the present invention, it has been discovered that a vascular access port can be made easier to use by one trying to insert a needle in such graft. This can be accomplished by inclusion within or on the port of a composition which absorbs UV light and fluoresces, i.e. a UV fluorescing composition, upon exposure to a source of UV light, such as a black light.

By exposing the area where the port is implanted to a black light the healthcare worker attempting to access the port will be able to see the exposed portion of the port, for example an access point or even see the port fluorescing under the skin for finding an access point.

Accordingly, in one embodiment of the invention, there is provided a vascular access port, at least a portion of which comprises a biocompatible UV fluorescing material, wherein the UV fluorescing material is positioned in the port such that, upon exposure to a UV light source, the port or a portion of the port subintimally implanted fluoresces sufficiently to improve the visibility of the location of the graft to a health care worker attempting to access the port.

In yet another embodiment of the present invention there is disclosed a method for a health care worker to access a vascular access port subintimally implanted in a patient comprising:
a) selecting a vascular access port at least a portion of which comprises a UV fluorescing material wherein the UV fluorescing material is positioned such that, when the port is subintimally implanted, upon exposure to a UV light source, the port or a portion of the port fluoresces sufficiently to improve the visibility of the location of the port by the health care worker;
b) positioning the port subintimally in the patient;
c) applying a UV light source to the general area where the port is positioned; and
d) identifying an access point in the port by observing the fluorescing port.

In yet another embodiment of the invention there is disclosed a method for a health care worker to access a vascular access port subintimally implanted in a patient comprising:
a) selecting a patient having a subintimally implanted vascular access port at least a portion of which comprises a UV fluorescing material, wherein the UV fluorescing material is positioned such that, upon exposure to UV light source, the port or portion of the port fluoresces sufficiently to improve visibility of the location of the port;
b) applying a UV light source to the area where the port is implanted; and
c) identifying a place to access the port by observing the fluorescing port.

### BRIEF DESCRIPTION OF THE DRAWNGS

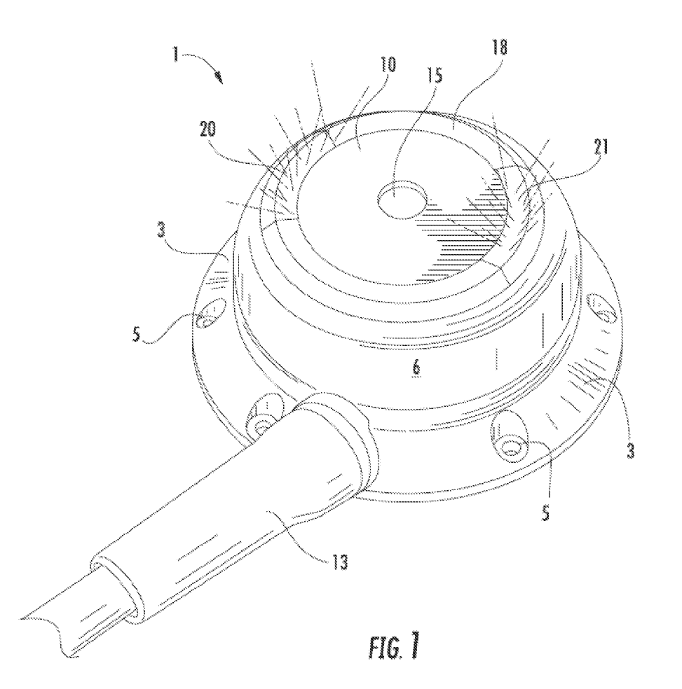

FIG. 1 is a perspective view of an embodiment of the present invention where there are two bands of UV fluorescing compound.

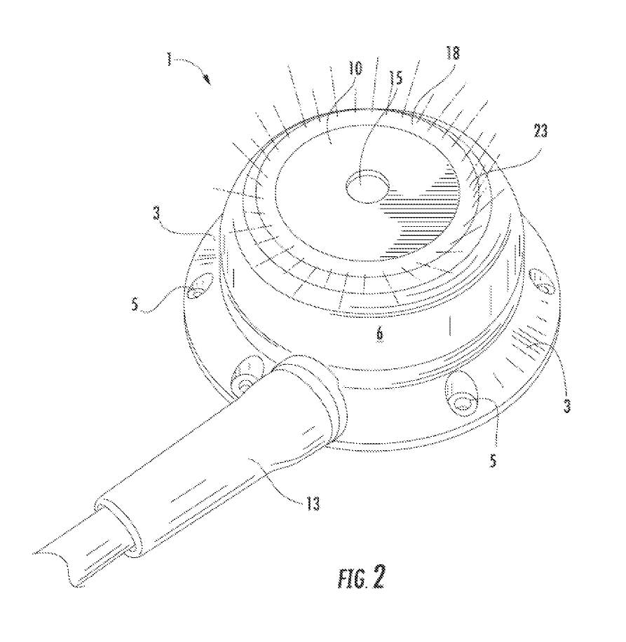

FIG. 2 is a perspective view of an embodiment of the present invention where there is a port with a ring entirely made with a single UV fluorescing polymer.

### DETAILED DESCRIPTION OF THE INVENTION

The present invention relates to the inclusion of a material which fluoresces upon application of UV light in at least a portion of a vascular access port. The present invention can include just around the injection sites or the entire device, which can be totally or partially under the skin during use. It can be the portion closest to the skin or any portion as desired. The present invention overcomes the limitations and problems of the prior art for those medical technicians attempting to insert a needle or other device into a vascular access port. Even though the problem has been around since the introduction of vascular access ports and still exists, and technology moves at a rapid pace, no previous solution accomplishes the results of the present invention.

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one as or more than one. The term “plurality”, as used herein, is defined as two, or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein “vascular access port” is a biocompatible device which is placed subintimally and usually attached by sutures to the underlying fascia. They are designed for adding or taking away fluids to/from the vasculature where multiple access is required to the patient, for example, during chemo treatment of cancer. A healthcare worker would use the port rather than continually inject or add new injection sites. The devices comprise an injection port for adding or taking a fluid away, a chamber and a tube which is in fluid communication with the chamber and a patient's vasculature. Placement of the device is where the access point is above or just under the skin making the port difficult to find by the healthcare worker. The biocompatible vascular access port can be made of a biocompatible polymer or metal which are within the skill in the art.

Medicaments, blood, neutrients or other material can be added or taken away from a patients vasculature by inserting a needed in to the port access hole and injecting or withdrawing fluid. It is clear that insertion of a needle through the skin and into the port should be as accurate as possible each time because of the problems associated with poor needle insertion which can cause infection and a whole host of other complications as described above.

As used herein, the term biocompatible UV fluorescing material relates to a biocompatible material which can be incorporated in, coated on or used to make a vasculature access port of the present invention. These compositions are photo-chromic substances, which are known to be essentially colorless but have the property of photo-reacting to longwave ultraviolet (UV) light (about 250 to 400 nm) and can change to a variety of colors and shades. The intensity of the resulting visible color (reds, violets, blues, etc) is directly proportional to the intensity of the UV light source, i.e. the more intense the UV light the more intense the resulting visible light will be.

One method of producing the present invention is to incorporate a photochromic compound directly into the polymer or other matrix making up the port. The polymer can be injection molded or the like directly into the port shape from there. Examples of plastic which could incorporate the compound for photochromic behavior include polyol(allyl carbonate)-monomers, polyacrylated, polyethylenes, polypropylenes, polyvinyl chloride, polymethylmethacrylates, cellulose acetate, cellulose triacetate, cellulose acetate propionate, cellulose acetate butyrate, polyacetal resins, acetyl cellulose, poly vinyl acetate, poly vinyl alcohols, poly urethanes, poly carbonates, polystyrenes, including copolymers and other biocompatible polymer molecules. The color of the fluorescing material will depend on the photochromic composition selected for inclusion in the present invention.

Another means of preparing the present invention is to incorporate the photochromic compound in one polymer and bind the polymer to the polymer or metal of the vascular access port. That way a particular area could be caused to glow and not just the entire vascular access port itself. While the photochromic compound could be included in just a portion of the fabrication material, separate polymer containing photochromic polymers (using the same or different polymers, metals or other biocompatible materials) would be easier to make. In one embodiment, only the area that around where a needle is to be inserted will glow. In another embodiment two sites, one on each side of the injection site could be caused to glow. It is clear the same or different colors could be used if multiple positions are cased to glow.

As used herein, the phrase, “UV light source” would refer to a longwave UV light, in one embodiment hand held, of sufficient light intensity to cause the UV polymer to glow considering its position subcutaneously, and still be able to visually observe the UV glow of the photochromic compound beneath the skin. Such UV handheld devices are well known within the art and are mentioned here for convenience.

In the use of the present invention, a patient in need of a vascular access port would have a vascular access port of the present invention surgically implanted and positioned in an appropriate place subintimally and sutured to the underlying fascia by a healthcare worker or technician. Once a vascular access port of the present invention is positioned in place in a patient, the healthcare worker would turn on a high intensity UV light and shine it in the general area (an arm or leg for example) where a vascular access port was placed and look for the appropriate glow. The worker could then, while observing the glow, insert the appropriate needles into the vascular access port for use in the patient. In one embodiment, where multiple vascular access ports are used, each site for needle insertion is color coded a different glowing color so that placement of each needle can easily be identified by use of separate colors of glowing polymer for each position.

Now, referring to the drawings, FIG. 1 is a perspective view of an embodiment of a UV glowing vascular access port 1. The vascular access port 1 is positioned subintimally and fastened to the underlying fascia by suturing the port 1 by using suture holes 5 which are in base 3. In this view, the outer wall of chamber 6 has top 10. Top 10 has in the center access point 15 for insertion of a needle or the like. The outer ring 18 is the outer edge of top 10. In this view, left portion 20 and right portion 21 of ring 18 are made of UV glowing material. This is shown as one type of UV material but in other embodiments could be different colors for 20 and 21 respectively. The port also has tubing 13 which extends to the vasculature as desired. The UV polymer in this embodiment is depicted as glowing but would need to have a UV source of light shined on the patient for the bands to continue to glow. The bands could also be reinforced as needed since it is intended that there will be multiple needle sticks into this region of the vascular access port.

FIG. 2 is a perspective view of another vascular access port 1 of the present invention. In this perspective a vascular access port 1 has the outer ring 18 made entirely of UV glowing polymer 23 such that upon exposure to the UV light, the entire outer ring 18 will glow. In this embodiment it would likely be that a single color would be impregnated or coated into the port outer ring 18 used for the vascular access port. Other features known for other vascular access ports could be included as well; however, the main feature of UV glowing polymer would remain the same. One skilled in the art in view of this disclosure could easily pick colors of polymers that fluoresce as well as means of combining multiple colors and colors at particular locations in the vascular access port. The disclosure in the specification and the claims which follow the specification are to be read broadly and not intended to be limited by any specific example or embodiment herein. While the outer top of the port shown with the fluorescing polymer, the port could have such polymer placed at any desirable position.

As can be seen from the embodiments above as well as in the figures, various embodiments can clearly be chosen varying the color location and the like of the UV polymer in the vascular access port of the present invention. Variations other than those embodiments described are within the skill of the art in view of the disclosure, and those such embodiments are within the scope of the claims which follow.

What is claimed:

1. A vascular access port, at least a portion of which comprises a biocompatible UV fluorescing material, wherein the UV fluorescing material is positioned in the port such that, upon exposure to a UV light source, the port or a portion of the port subintimally implanted fluoresces sufficiently to improve the visibility of the location of the graft to a health care worker attempting to access the port.

2. A port according to claim 1 where the UV fluorescing material is positioned at one or more port access sites.

3. A port according to claim 2 wherein there is UV fluorescing material positioned at least two sites and wherein each site uses a different color of fluorescing material.

4. A port according to claim 1 wherein the UV fluorescing material is a biocompatible UV fluorescing polymer.

5. A port according to claim 1 wherein a UV fluorescing compound is admixed with the composition of the port.

6. A method for a health care worker to access a vascular access port subintimally implanted in a patient comprising:
a) selecting a vascular access port at least a portion of which comprises a UV fluorescing material wherein the UV fluorescing material is positioned such that, when the port is subintimally implanted, upon exposure to a UV light source, the port or a portion of the port fluoresces sufficiently to improve the visibility of the location of the port by the health care worker;
b) positioning the port subintimally in the patient;
c) applying a UV light source to the general area where the port is positioned; and
d) identifying an access point in the port by observing the fluorescing port.

7. A method for a health care worker to access a vascular access port subintimally implanted in a patient comprising:
a) selecting a patient having a subintimally implanted vascular access port at least a portion of which comprises a UV fluorescing material, wherein the UV fluorescing material is positioned such that, upon exposure to UV light source, the port or portion of the port fluoresces sufficiently to improve visibility of the location of the port;
b) applying a UV light source to the area where the port is implanted; and
c) identifying a place to access the port by observing the fluorescing port.
